(19) United States
(12) Patent Application Publication
(10) Pub. No.: US 2026/0103726 A1
(43) Pub. Date: Apr. 16, 2026

(54) CONTROL SYSTEM FOR INDUCING TRANSGENE EXPRESSION BY MEANS OF D-2-HG, AND CONSTRUCTION METHOD THEREFOR AND USES THEREOF

(71) Applicant: THE SECOND HOSPITAL OF SHANDONG UNIVERSITY, Jinan, Shandong (CN)

(72) Inventors: Wen ZHANG, Jinan (CN); Mu YANG, Jinan (CN); Fang WANG, Jinan (CN); Dongqi TANG, Jinan (CN); Jiang LIU, Jinan (CN)

(73) Assignee: THE SECOND HOSPITAL OF SHANDONG UNIVERSITY, Jinan, Shandong (CN)

(21) Appl. No.: 18/995,217

(22) PCT Filed: Jan. 10, 2024
(86) PCT No.: PCT/CN2024/071496

§ 371 Date: Jan. 16, 2025

(30) Foreign Application Priority Data
Feb. 1, 2023 (CN) 202310085103.1

(51) Int. Cl. C12N 15/86; C12N 15/85

(52) U.S. Cl. CPC C12N 15/86; C12N 15/85; C12N 2740/15043; C12N 2800/107; C12N 2830/002

(57) ABSTRACT

A control system for inducing transgene expression by means of D-2-hydroxyglutarate (D-2-HG), which relates to the technical fields of synthetic biology, gene therapy and cell immunity. A sensing object of the control system is D-2-HG; and the control system is classified into a control system for inducing transgene expression by means of high-concentration D-2-HG (HGind-H) and a control system for inducing transgene expression by means of low-concentration D-2-HG (HGind-L). The control system includes a recombinant transcriptional repressor, a D-2-HG inducible promoter and a sequence to be transcribed. According to the present invention, the system is used to control a diagnostic gene circuit, a suicide gene circuit and an immune cell therapeutic gene circuit that respond to a tumor metabolite D-2-HG, thereby developing living cell sensors, suicide gene therapy products and cell therapy products with D-2-HG as key information.

### REFERENCE TO AN ELECTRONIC SEQUENCE LISTING

The present application contains a Sequence Listing that has been submitted electronically and is hereby incorporated by reference in its entirety. The electronic Sequence Listing is named Sequence_Listing_ST26, was created on Dec. 19, 2025, and is 36,183 bytes in size.

### TECHNICAL FIELD

The present invention relates to the technical fields of synthetic biology, gene therapy and cell immunity, and particularly relates to a control system for inducing transgene expression by means of D-2-HG and a construction method therefor and uses thereof.

### BACKGROUND

In recent years, it has been discovered that D-2-hydroxyglutarate (D-2-HG) is a tumor metabolite. IDH (isocitrate dehydrogenase) mutations exist in a variety of tumor cells, IDH is a key enzyme in a tricarboxylic acid cycle, and its original function is to convert isocitrate into 2-ketoglutarate (2-KG). However, mutations can cause the enzyme to produce a strong activity in catalyzing the reduction of 2-KG to D-2-HG, resulting in the accumulation of D-2-HG in cells and tumor microenvironments. Therefore, such metabolite D-2-HG is an important entry point for the development of tumor treatment strategies and drugs in the future. For example, T cells can be engineered to sense the concentration of D-2-HG and thus exert anti-tumor functions; and suicide gene circuits that sense D-2-HG can be explored to cause specific suicide of tumor cells.

As an alternative marker for IDH, D-2-HG has shown a great use value in distinguishing IDH mutations in tumors such as gliomas and acute myeloid leukemia. Early diagnosis and sensitive monitoring of recurrence/metastasis are the key to effectively treat tumors. However, current IDH detection methods face the problems as follows: fewer markers produced in early tumors; and the transport restrictions, massive dilution and rapid discharge and the like of markers from a microenvironment into a circulatory system. Chemical molecular probes used in vivo are difficult to accurately reach tumor sites by passive diffusion. A “living” sensor based on engineered cells can not only detect the concentration of D-2-HG in vitro, but also actively migrate to the tumor in vivo, sense tumor information and amplify signals, which represents an emerging high-sensitivity tumor diagnosis technology. For example, some researchers have modified macrophages to drive genes of luciferase expression through a promoter of arginase-1. These engineered macrophages after being injected into a body migrate to tumor sites and activate the production of luciferase; they can detect tumors with a volume of 25-50 mm³, and their sensitivity is higher than that of proteins and nucleic acid marker detection methods used in clinical practices. The success of engineered immune cell drugs such as CAR-T has opened the way for the future clinical application of living cells. D-2-HG accumulated in the microenvironment, as the specific information of IDH mutant tumors, is an ideal sensing object for this “living” sensor.

Therefore, there is an urgent need to develop therapeutic products or cell sensors with D-2-HG as information, which effectively designs, optimizes and assembles functional components of different organisms, and carries out targeted transformation on cells so that their specific functions meet specific needs.

### SUMMARY

In order to solve the problem that specific needs cannot be met by means of products sensing a tumor metabolite D-2-HG at present, an objective of the present invention is to provide a control system for inducing transgene expression by means of D-2-HG and a construction method therefor and uses thereof.

In order to achieve the above objective, the present invention is implemented through the following technical solutions:

A control system for inducing transgene expression by means of D-2-hydroxyglutarate (D-2-HG) is provided; a sensing object of the control system is D-2-HG, and the control system is classified into a control system for inducing transgene expression by means of high-concentration D-2-HG (HGind-H) and a control system for inducing transgene expression by means of low-concentration D-2-HG (HGind-L); the control system comprises a recombinant transcriptional repressor, a D-2-HG inducible promoter and a sequence to be transcribed.

The high concentration is greater than 0.5 mmol/L, and the low concentration is less than or equal to 0.5 mmol/L.

Preferably, the recombinant transcriptional repressor is obtained by fusing a transcriptional repressor protein KRAB, a bacterial transcription repression regulatory factor DhdR that senses D-2-HG, and a nuclear localization signal NLS;

the transcriptional repressor protein KRAB is a rat zinc-finger protein Kid-1, the sequence of which is SEQ ID NO.1, or a human zinc-finger protein ZNF10, the sequence of which is SEQ ID NO.2;

the bacterial transcription repression regulatory factor DhdR that senses D-2-HG is a bacterial transcription repression regulatory protein that responds to D-2-HG, and should meet the following features:

DhdR has a DNA binding domain and a ligand binding domain, and in bacteria, DhdR binds to a DhdR protein-specific binding DNA sequence DhdO to prevent the transcription of a target gene, and after binding to D-2-HG, dissociates from DhdO, so that the target gene is transcribed, and DhdR and DhdO constitute a bacterial D-2-HG operon; and

DhdR may be DhdR-AD in Achromobacter denitrificans NBRC 15125 (sequence: SEQ ID NO. 3), DhdR-AX in Achromobacter xylosoxidans ATCC27061 (sequence: SEQ ID NO. 4) or other bacterial transcription repression regulatory factors that meet the above features.

In the recombinant transcriptional repressor, the KRAB can be located at an N-terminus or C-terminus of DhdR.

The nuclear localization signal NLS is a domain that guides a protein into a cell nucleus, and includes, but is not limited to, a NLS derived from a SV40 large T antigen, and the amino acid sequence of the NLS is PKKKRKV.

Preferably, the D-2-HG inducible promoter is formed by tandem connection of a constitutive promoter Pc and a DhdR protein-specific binding DNA sequence DhdO, DhdO being located upstream or/and downstream of Pc; and the D-2-HG inducible promoter is abbreviated as DhdO(n₁)-Pc-DhdO(n₂), wherein n₁ and n₂ are the numbers of tandem repeats of DhdO, 0≤n₁≤14, 0≤n₂≤14, and n₁ and n₂ are not 0 simultaneously; and

Pc is a conventional promoter for constitutive expression in a eukaryotic cell, specifically CMV, hPGK, mPGK or EF1α.

The minimum sequence of DhdO is GTTATCAGATAAC; and to increase or decrease the binding capability of DhdO to DhdR, point mutations can also be introduced based on this sequence.

Preferably, the sequence to be transcribed comprises a reporter gene sequence and a protein or small peptide gene sequence that can be used as a treatment for disease, wherein the reporter gene sequence comprises, but is not limited to, secretory alkaline phosphatase, Gaussia luciferase, firefly luciferase, and an enhanced fluorescent protein, and the gene sequence that can be used as a treatment for disease includes, but is not limited to, a chimeric antigen receptor, a cytokine, a chemokine, a suicide gene or a D-2-HG catabolic enzyme.

Preferably, when the control system is a control system for inducing transgene expression by means of low-concentration D-2-HG (HGind-L), the control system further comprises a D-2-HG transporter protein;
the D-2-HG transporter protein is a protein that transports D-2-HG into a cell in mammalian cells, such as a SLC13A3 protein, and the SLC13A3 protein is driven to express by a weak promoter or minimal promoter; and the weak promoter or the minimal promoter comprises, but is not limited to, a minimal CMV promoter, a mini-TK promoter or CMV53.

The concentration of D-2-HG in the present invention can be artificially added, or formed by an organism itself. The reasons for the formation by the organism itself include, but are not limited to, gene mutations of isocitrate dehydrogenase (IDH), enhanced expression of hydroxyacid-oxoacid transhydrogenase (ADHFE1), and gene mutations of D-2-hydroxyglutarate dehydrogenase (D2HGDH); and the concentration can be a concentration of D-2-HG in body fluids, cells, tumor microenvironment, etc.

The present invention further comprises a construction method for the control system for inducing transgene expression by means of D-2-HG, which comprises the following steps:
(1) a control system for inducing transgene expression by means of D-2-HG was designed and synthesized, wherein the system comprises a recombinant transcriptional repressor, a D-2-HG inducible promoter, and a sequence to be transcribed;
(2) a vector carrying the control system for inducing transgene expression by means of D-2-HG was prepared, wherein the vector is a eukaryotic plasmid expression vector, a viral particle or a transfection reagent;
(3) the vector carried the control system for inducing transgene expression by means of D-2-HG to enter a target cell, wherein the target cell is a cell line, a tumor cell or an immune cell; and
(4) the target cell sensed the concentration of D-2-HG and then induced transgene expression.

Preferably, in the construction method, when the control system is a control system for inducing transgene expression by means of low-concentration D-2-HG (HGind-L), when a control system for inducing transgene expression by means of D-2-HG was designed and synthesized at step 1, a D-2-HG transporter protein needs to be added.

The present invention further comprises application of the control system for inducing transgene expression by means of D-2-HG in constructing a D-2-HG living cell sensor, and application of the living cell sensor in vitro and in vivo.

The present invention further comprises application of the control system for inducing transgene expression by means of D-2-HG in constructing a suicide gene therapy vector and application of the suicide gene therapy vector in a tumor suicide gene therapy.

The present invention further comprises application of the control system for inducing transgene expression by means of D-2-HG in constructing therapeutic cells and application of the therapeutic cells in tumor treatment.

Compared with the prior art, the present invention has the following advantages:

The development of small molecule inhibitor drugs targeting tumor IDH mutations is the main strategy of the current precision medicine direction. The present invention is not aimed at IDH gene mutations, but based on a key feature of D-2-HG accumulation in tumor cells and microenvironments. According to the present invention, a control system for inducing transgene expression by means of D-2-HG is established firstly, and then the system is used to control a diagnostic gene circuit, a suicide gene circuit and an immune cell therapeutic gene circuit that respond to a tumor metabolite D-2-HG, so that living cell sensors, suicide gene therapy products and cell therapy products with D-2-HG as key information are developed.

The control system for inducing transgene expression by means of D-2-HG according to the present invention is classified into a high concentration and a low concentration, where the high-concentration control system is composed of a recombinant transcriptional repressor, a D-2-HG inducible strong promoter and a sequence to be transcribed, where the gene sequence to be transcribed is a reporter gene, a suicide gene or a therapeutic gene, and can be used to develop living cell sensors, suicide gene therapy products or cell therapy products; the low-concentration control system is composed of a recombinant transcriptional repressor, a D-2-HG transporter protein, a D-2-HG inducible strong promoter and a sequence to be transcribed, where the gene sequence to be transcribed is a reporter gene, and is particularly suitable for developing high-sensitivity living cell sensors.

### BRIEF DESCRIPTION OF THE DRAWINGS

FIG. 1 is a schematic composition diagram of a control system for inducing transgene expression by means of D-2-HG;

FIG. 2 is an arrangement graph of KRAB and DhdR in a recombinant transcriptional repressor;

FIG. 3 is a diagram showing an optimization of two-plasmid ratio in HGind composed of the two plasmids;

FIG. 4 is a diagram showing an optimization of the number of tandem connection of DhdOs in HGind composed of two plasmids;

FIG. 5 is a schematic diagram showing results of D-2-HG dose-responsive induction of D2eGFP expression in HGind composed of two plasmids;

FIG. 6 is a schematic diagram showing a HGind response of introduction of SLC13A3 to low-concentration D-2-HG;

FIG. 7 is a schematic diagram showing an effect of optimizing an addition amount of SLC13A3 expression plasmids in a system on gene expression;

FIG. 8 is a schematic diagram showing a response status of HGind-L composed of three plasmids to D-2-HG;

FIG. 9 is a schematic diagram of a composition structure of a long fragment A formed by whole gene synthesis;

FIG. 10 is a schematic diagram showing effects of different concentrations of GCV on cytotoxicity;

FIG. 11 is a schematic diagram showing tumor sizes of mice in Saline (n=8), GCV (n=10) and saline groups;

FIG. 12 is a schematic diagram showing tumor weights of mice in each group (HT1080-saline, HT1080-GCV, HT1080-DHDO14-saline, HT1080-DHDO14-GCV); and

FIG. 13 is a schematic diagram showing tumor volumes of mice in each group (HT1080-saline, HT1080-GCV, HT1080-DHDO14-saline, HT1080-DHDO14-GCV).

### DETAILED DESCRIPTION OF EXAMPLES

An objective of the present invention is to provide a control system for inducing transgene expression by means of D-2-HG, and a construction method therefor and uses thereof, which are achieved through the following technical solutions:

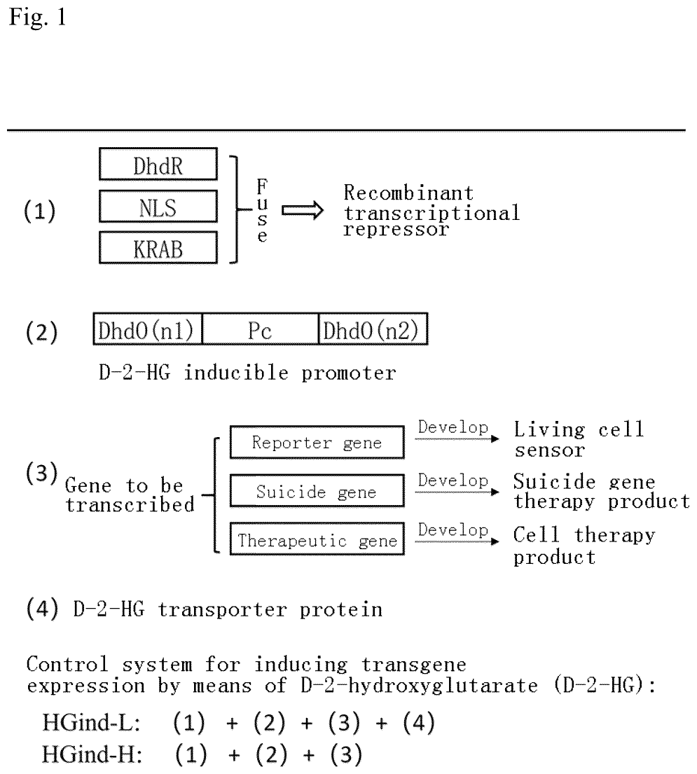

I. A control system for inducing transgene expression by means of D-2-hydroxyglutarate (D-2-HG) is provided; a sensing object of the control system is D-2-HG, and the control system is classified into a control system for inducing transgene expression by means of high-concentration D-2-HG (HGind-H) and a control system for inducing transgene expression by means of low-concentration D-2-HG (HGind-L); as shown in FIG. 1:

### (I) Control System for Inducing Transgene Expression by Means of High-Concentration D-2-HG (HGind-H)

The control system for inducing transgene expression by means of high-concentration D-2-HG (HGind-H) includes a recombinant transcriptional repressor, a D-2-HG inducible promoter, and a sequence to be transcribed. The high-concentration D-2-HG is D-2-HG with a concentration of greater than 0.5 mmol/L.

### (II) Control System for Inducing Transgene Expression by Means of Low-Concentration D-2-HG (HGind-L)

The control system for inducing transgene expression by means of low-concentration D-2-HG (HGind-L) includes a recombinant transcriptional repressor, a D-2-HG inducible strong promoter and a sequence to be transcribed, and preferably further includes a D-2-HG transporter protein. The low-concentration D-2-HG is D-2-HG with a concentration of less than or equal to 0.5 mmol/L.

The recombinant transcriptional repressor is formed by fusing a transcriptional repressor protein KRAB (Krueppel-associated box protein), a bacterial transcription repression regulatory factor (named DhdR) that senses D-2-HG, and a nuclear localization signal (NLS). The transcriptional repressor protein KRAB can be derived from a rat zinc-finger structural protein Kid-1, the sequence of which is SEQ ID NO.1, or can be derived from a human zinc-finger structural protein ZNF10 (abbreviated as h-KRAB), the sequence of which is SEQ ID NO.2.

DhdR has the following features: DhdR contains a DNA binding domain and a ligand binding domain, and in bacteria, DhdR binds to a DNA binding sequence (DhdO) to prevent the transcription of a target gene, and after binding to D-2-HG, dissociates from DhdO, so that the target gene is transcribed. DhdR may be DhdR-AD in Achromobacter denitrificans NBRC 15125 (sequence: SEQ ID NO. 3), DhdR-AX in Achromobacter xylosoxidans ATCC27061 (sequence: SEQ ID NO. 4) or other bacterial transcription repression regulatory factors that meet the above features.

NLS is a domain that guides a protein into a cell nucleus. The most commonly used one is a NLS derived from a SV40 large T antigen. The amino acid sequence of this NLS is PKKKRKV.

In the recombinant transcriptional repressor, KRAB can be located at an N-terminus or C-terminus of DhdR. DhdR-AD, NLS and KRAB derived from Kid-1 are fused into DhdR-AD-KRAB (sequence: SEQ ID NO.5) or KRAB-AD-DhdR (sequence: SEQ ID NO.6). DhdR-AX, NLS and h-KRAB are fused into DhdR-AX-hKRAB (sequence: SEQ ID NO.7).

The D-2-HG inducible promoter is formed by tandem connection of a constitutive promoter (Pc) and a specific DNA binding sequence (DhdO), where DhdO is located upstream or/and downstream of Pc; and the D-2-HG inducible promoter is abbreviated as DhdO(n₁)-Pc-DhdO(n₂), where n₁ and n₂ are the numbers of tandem repeats of DhdO, 0≤n₁≤14, 0≤n₂≤14, and n₁ and n₂ are not 0 simultaneously; and

Pc is a conventional promoter for constitutive expression in a eukaryotic cell, such as CMV, hPGK, mPGK, and EF1α. The minimum sequence of DhdO is GTTATCAGATAAC. In addition, in order to increase or decrease the binding capability of DhdO to DhdR, point mutations can also be introduced based on DhdO sequence [Nature Communications. 2021, 12:7108.].

The sequence to be transcribed includes a reporter gene sequence and a protein or small peptide gene sequence that can be used as a treatment for disease, where the reporter gene sequence includes secretory alkaline phosphatase, Gaussia luciferase, firefly luciferase, and an enhanced fluorescent protein, and the gene sequence that can be used as a treatment for disease includes a chimeric antigen receptor, a cytokine, a chemokine, a suicide gene, a D-2-HG catabolic enzyme, etc.

The D-2-HG transporter protein is a protein that transports D-2-HG into mammalian cells. In the present application, SLC13A3 is selected, and its sequence is SEQ ID NO.8; and SLC13A3 is preferably driven to express by a weak promoter or minimal promoter; the weak promoter or minimal promoter includes, but is not limited to, a common minimal CMV promoter (sequence: SEQ ID NO.9), a mini-TK promoter (sequence: SEQ ID NO.10), a CMV53 (sequence: SEQ ID NO.11), etc.

### II. Use of the Control System for Inducing Transgene Expression by Means of D-2-HG in Constructing a Suicide Gene Therapy Vector

For tumor cells with IDH mutations and ADHFE1 overexpression, the accumulation of high-concentration D-2-HG inside the cells is a typical feature that distinguishes these cells from normal cells. An enzyme encoded by a suicide gene (Sui) can catalyze conversion of non-toxic drug precursors into cytotoxic substances, thereby causing a recipient cell carrying the gene to be killed. Suicide genes can be selected from herpes simplex virus thymidine kinase (HSV-TK) or cytosine deaminase (CD) gene, and corresponding drug precursors thereof are ganciclovir or 5-fluorocytosine, respectively.

A construction method includes the following steps: (1) A control system for inducing transgene expression by means of D-2-HG was designed and synthesized: a sequence to be transcribed in HGind-H was selected as a suicide gene to form a suicide gene circuit (HGind-H-Sui) that senses D-2-HG. (2) A vector carrying HGind-H-Sui was prepared, where the vector is a eukaryotic plasmid expression vector, a viral particle such as a lentivirus or an oncolytic virus, or a transfection reagent such as a polymer. (3) The vector carried HGind-H-Sui to enter a target cell, where the target cell can be a cell line or a primary cell such as a tumor cell. (4) The target cell sensed a concentration of D-2-HG and expressed the suicide gene, thereby inducing cell suicide.

### III. Use of the Control System for Inducing Transgene Expression by Means of D-2-HG in Constructing a Living Cell Biosensor and Use of the Living Cell Sensor In Vitro and In Vivo

The living cell sensors can respond to the concentration of D-2-HG in vitro, and can also be implanted in animals to sense the concentration of D-2-HG in the blood; and the living cell sensors based on macrophages or immune cells can migrate to tumor sites for evaluation and detection of related tumors.

A construction method for a living cell sensor includes the following steps: (1) A control system for inducing transgene expression by means of D-2-HG was designed and synthesized. A reporter gene is selected as a sequence to be transcribed. The reporter gene can be selected from secretory alkaline phosphatase (SEAP), firefly luciferase, Gaussia luciferase, etc. For sensors used in vivo, Gaussia luciferase is preferred because it has the advantages of secretability, high luminous intensity, no need for ATP, short half-life, and suitability for real-time monitoring of living cells or organisms. Depending on the sensitivity requirements of the sensors, HGind-H or HGind-L can be selected, so that a gene circuit that senses high-concentration or low-concentration D-2-HG is formed. (2) A vector carrying the control system for inducing transgene expression by means of D-2-HG was prepared, where the vector is a eukaryotic plasmid expression vector, a viral particle such as lentivirus, or a transfection reagent such as a polymer. (3) The vector carried the control system for inducing transgene expression by means of D-2-HG to enter a target cell, where the target cell can be a cell line and a primary cell such as an immune cell. (4) These living cell sensors sense the concentration of D-2-HG and thus induce reporter gene expression.

### IV. Use of the Control System for Inducing Transgene Expression by Means of D-2-HG in Constructing Therapeutic Cells and Use of the Therapeutic Cells in Tumor Treatment

For solid tumors with IDH mutations and ADHFE1 overexpression, high-concentration D-2-HG is accumulated in their tumor microenvironments. A construction method for the therapeutic cells includes the following steps: (1) A control system for inducing transgene expression by means of D-2-HG was designed and synthesized. For constructing the therapeutic cells, a sequence to be transcribed should be a therapeutic gene. The therapeutic gene can be the expression of genes such as a chimeric antigen receptor (CAR), a chemokine, a cytokine, an antibody, and a D-2-HG catabolic enzyme. (2) A vector carrying the control system for inducing therapeutic gene expression by means of D-2-HG was prepared, where the vector is a eukaryotic plasmid expression vector, a viral particle such as a lentivirus, or a transfection reagent such as a polymer; (3) the vector carried the control system for inducing transgene expression by means of D-2-HG to enter the target cell, where the target cell can be an immune cell such as a T lymphocyte, a NK cell, or other mammalian source cells. (4) These therapeutic cells sense the concentration of D-2-HG and thus induce therapeutic gene expression.

The present invention is further described below in conjunction with specific examples.

### Example 1

A construction process of HGind-H consisting of two plasmids includes the following steps:

(1) Whole gene synthesis of a recombinant transcriptional repressor: based on amino acid sequences, two fusion genes, i.e., DhdR-AD-KRAB whose sequence is SEQ ID NO.5 (KRAB was located at a C-terminus) and KRAB-AD-DhdR whose sequence is SEQ ID NO.6 (KRAB was located at an N-terminus) were linked into pCDNA3.1(+) through HindIII/KpnI restriction enzyme cutting sites respectively to construct plasmids pCDNA3.1(+)-DhdR-KRAB and pCDNA3.1(+)-KRAB-DhdR, and the recombinant transcriptional repressor was driven to express by a CMV promoter of pCDNA3.1(+).

(2) A fragment (having a sequence of SEQ ID NO.12 and containing BamHI at a 5′ terminus and XbaI restriction enzyme cutting sites at a 3′ terminus) of 10 tandemly repeated DhdOs (DhdO10) and a fluorescent protein D2eGFP is formed by whole gene synthesis, the fragment was named DhdO10-D2eGFP, which was linked into pCDNA3.1(+) through BamHI and XbaI to construct a plasmid pCDNA3.1(+)-DhdO10-D2eGFP on which Dhd010 and CMV constituted a D-2-HG inducible promoter.

(3) The plasmid was transfected into HEK293FT cells with lipofectamine 3000; a transfection plasmid combination 1 was pCDNA3.1(+)-DhdR-KRAB and pCDNA3.1(+)-DhdO10-D2eGFP or a plasmid combination 2 was pCDNA3.1(+)-KRAB-DhdR and pCDNA3.1(+)-DhdO10-D2eGFP; 3*105/well of cells were seeded in a 12-well plate, pcDNA3.1(+)-DhdO10-D2eGFP was added at lug/well, and the ratio of the recombinant transcriptional repressor plasmid to PcDNA3.1(+)-DhdO10-D2eGFP was 0:1, 1:1, 3:1.

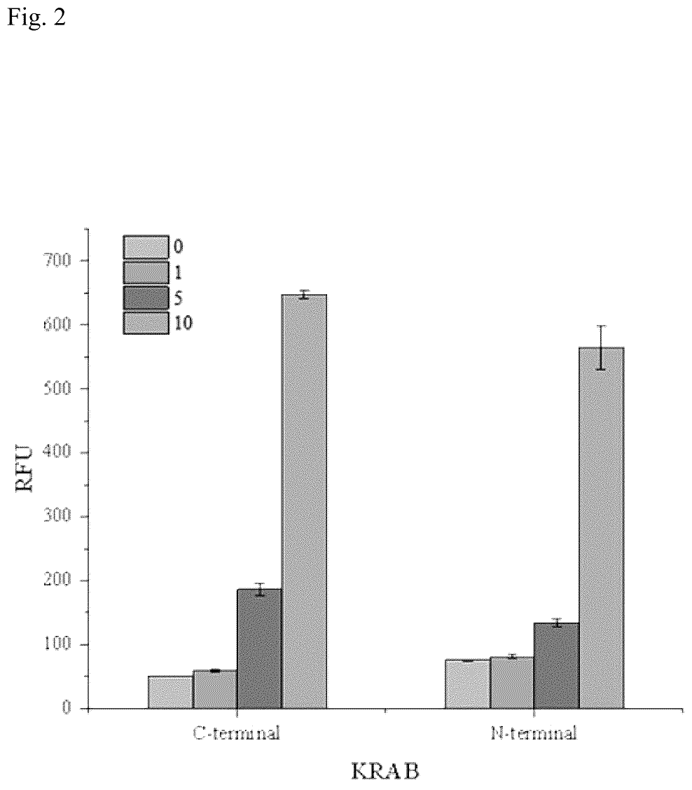

(4) On the next day, D-2-HG was added at concentrations of 0 mM, 1 mM, 5 mM, and 10 mM; 64 h after transfection, a fluorescence microscope was used to take pictures, the supernatant was aspirated, a fluorescence microplate reader measured fluorescence intensities, and results were shown in FIG. 2.

(5) FIG. 2 shows that the two plasmid combinations, i.e., pCDNA3.1(+)-DhdR-KRAB and pCDNA3.1(+)-DhdO10-D2eGFP, or pCDNA3.1(+)-KRAB-DhdR and pCDNA3.1(+)-DhdO10-D2eGFP can achieve that D-2-HG dosimetrically controls expression of D2eGFP.

### Example 2

An experiment on the ratio of two plasmids in the HGind-H system described in Example 1

(1) pCDNA3.1(+)-DhdR-KRAB and pCDNA3.1(+)-DhdO10-D2eGFP were selected, and then transfected using liposomes as above; the ratio of the two plasmids was set to 0, 0.1, 0.3, 0.4, 0.5, 0.75, 1.

(2) On the next day, D-2-HG was added at concentrations of 0 mM and 5 mM; and 64 h after transfection, a fluorescence microscopy was used to take pictures, the supernatant was aspirated, and a fluorescence microplate reader measured a fluorescence intensity.

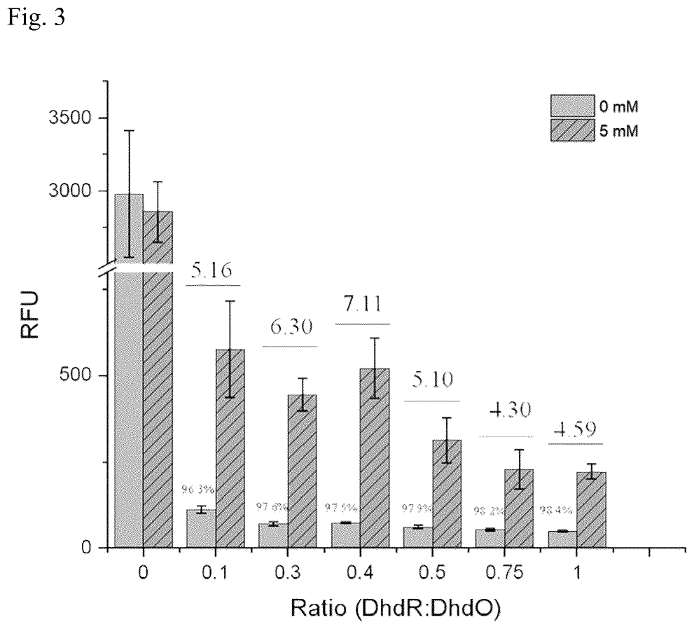

(3) As shown in FIG. 3, results showed that when the ratio was 0 (i.e., no pCDNA3.1(+)-DhdR-KRAB was added), the addition of 0 mM and 5 mM D-2-HG had little effect on the fluorescence intensity; as shown in Table 1, when the ratio was 0.1, the repression efficiency (compared with that when the ratio was 0) can reach 96.3%; as the ratio increases, the repression become stronger and stronger, and the repression efficiency reaches a maximum of 98.4% (when the ratio is 1); and the induction multiple (the fluorescence intensity when the concentration was 5 mM was divided by the fluorescence intensity when the concentration was 0 mM) reached the maximum (7.11 times) when the ratio was 0.4.

TABLE 1
Relationships between the ratio of two plasmids
in HGind composed of the two plasmids and the
repression efficiency and the induction multiple

| Ratio of two plasmids | Repression efficiency (%) | Induction multiple |
|---|---|---|
| 0 | / | / |
| 0.1 | 96.3 | 5.16 |
| 0.3 | 97.6 | 6.30 |
| 0.4 | 97.5 | 7.11 |
| 0.5 | 97.9 | 5.10 |
| 0.75 | 93.2 | 4.30 |
| 1 | 98.4 | 4.59 |

### Example 3

The number of tandem repeats of DhdO in the HGind-H system in Example 1

(1) A fragment of n (n=3 or 7 or 10 or 14) tandemly repeated DhdO (DhdOn) and a fluorescent protein D2eGFP (sequence: SEQ ID NO.12-15) was formed by whole gene synthesis, the fragment was named DhdOn-D2eGFP, which was linked into pCDNA3.1(+) through BamHI and XbaI to construct a plasmid pCDNA3.1(+)-DhdOn-D2eGFP on which DhdOn and CMV constituted a D-2-HG inducible promoter.

(2) The plasmid was transfected into HEK293FT cells with lipofectamine 3000; a transfection plasmid combination is pCDNA3.1(+)-DhdR-NLS-KRAB and pCDNA3.1(+)-DhdOn-D2eGFP; n=3 or 7 or 10 or 14.

(3) On the next day, D-2-HG was added at concentrations of 0 mM and 5 mM; and 64 h after transfection, a fluorescence microscopy was used to take pictures, the supernatant was aspirated, and a fluorescence microplate reader measured a fluorescence intensity.

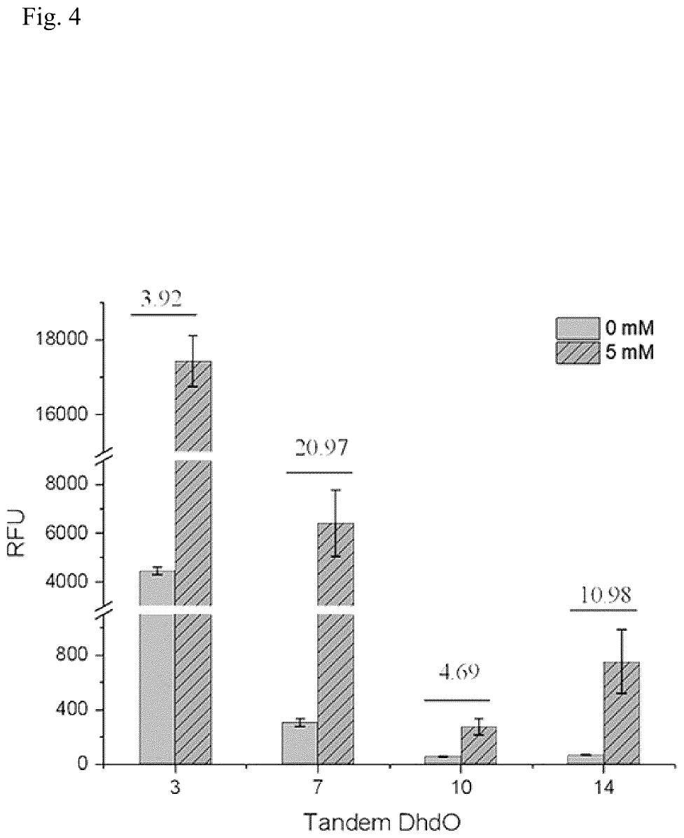

(4) As shown in FIG. 4, results showed that when n=3 or 7 or 10 or 14, the induction multiples were 3.92, 20.97, 4.69 and 10.98 respectively. Therefore, when n=7, the induction multiple is the largest.

### Example 4

D-2-HG dose-responsive induction of D2eGFP expression in the HGind-H system in Examples 1-3

(1) pCDNA3.1(+)-DhdR-KRAB and pCDNA3.1(+)-Dhd07-D2eGFP were selected, and then transfected using liposomes as above; and the ratio of the two plasmids was set to 0.4.

(2) On the next day, D-2-HG was added at concentrations of 0, 0.5, 1, 5, 10, and 20 mM; and a fluorescence microplate reader measured a fluorescence intensity.

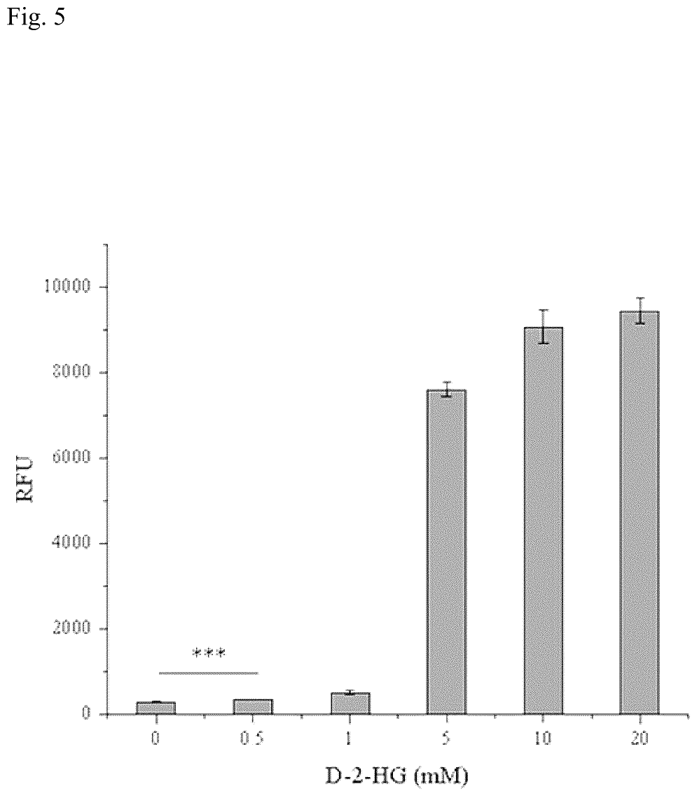

(3) As shown in FIG. 5, results showed that 0.5 mM D-2-HG can produce a significant difference from a 0 mM D-2-HG group; and as the concentration increases, the fluorescence intensity becomes larger and larger.

### Example 5

Effects of the introduction of a D-2-HG transporter protein SLC13A3 on the above system

(1) Based on an amino acid sequence (sequence: SEQ ID NO.8), SLC13A3 was formed by whole gene synthesis and cloned into a pCDNA3.1(+) vector to construct pCDNA3.1(+)-SLC13A3; at the same time, SEAP (sequence: SEQ ID NO.16) with a similar sequence length was synthesized and cloned into pCDNA3.1(+) vector to construct pCDNA3.1(+)-SEAP, this plasmid can be used as a control.

(2) In addition to the two plasmids pCDNA3.1(+)-DhdR-KRAB and pCDNA3.1(+)-DhdO7-D2eGFP, a third plasmid (i.e., pCDNA3.1(+)-SLC13A3 and pCDNA3.1(+)-SEAP constructed at step 1) was transfected using liposomes as above; the ratio of the three plasmids was set to 1:0.4:0.1.

(3) On the next day, D-2-HG was added at concentrations of 0, 0.05, 0.25, 0.5, 1, and 5 mM.

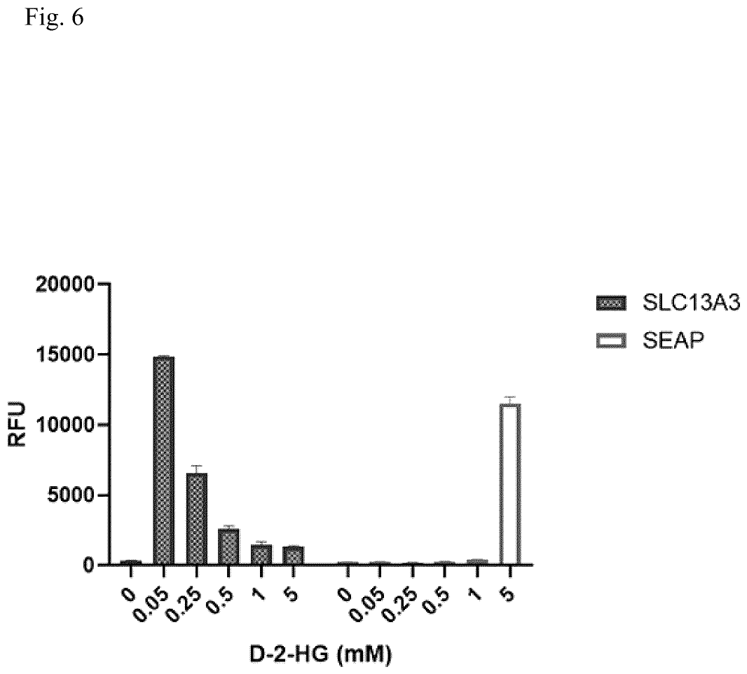

(4) Results were shown in FIG. 6, from which it can be seen that the addition of pCDNA3.1(+)-SEAP did not cause a significant change in D-2-HG induced transgene expression; while pCDNA3.1(+)-SLC13A3 allowed the response of the system to low-concentration D-2-HG (0-0.05 mM) to be greatly improved compared to the addition of pCDNA3.1(+)-SEAP.

### Example 6

### Optimization of the Addition Amount of an SLC13A3 Expression Plasmid in the System

(1) In addition to the two plasmids pCDNA3.1(+)-DhdR-KRAB and pCDNA3.1(+)-DhdO7-D2eGFP, a third plasmid, i.e., pCDNA3.1(+)-SLC13A3, was transfected using liposomes as above; the ratio of the three plasmids was set to 1:0.4:X; X=0 or 0.01 or 0.05 or 0.1 or 0.2 or 0.4.

(2) On the next day, D-2-HG was added at concentrations of 0 (control), 5, and 50 μM; and a fluorescence microplate reader measured a fluorescence intensity.

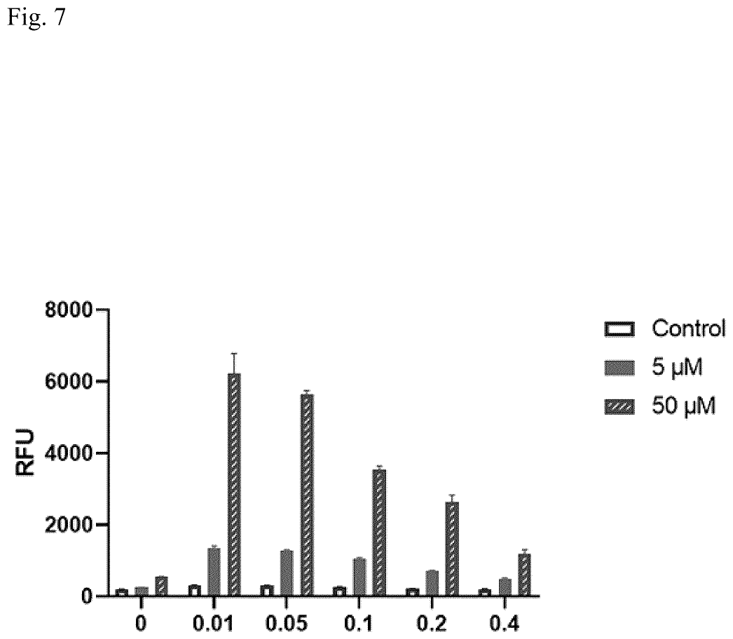

(3) Results were shown in FIG. 7, when the addition amount of pCDNA3.1(+)-SLC13A3 was the lowest (the ratio is 0.01), the fluorescence intensity was the highest, that is, the system induced the highest transgene expression. This also means that SLC13A3 can cause the sensitivity of the system to D-2-HG to be greatly enhanced without requiring a very high expression quantity.

### Example 7

### HGind-L Composed of Three Plasmids

(1) Three plasmids, i.e., pCDNA3.1(+)-DhdR-KRAB, pCDNA3.1(+)-DhdO7-D2eGFP and pCDNA3.1(+)-SLC13A3, respectively carried a recombinant transcriptional repressor, a D-2-HG inducible promoter and a D-2-HG transporter protein which constituted a control system for inducing transgene expression by means of low-concentration D-2-HG (HGind-L).

(2) Transfection was performed using liposomes as above; and the ratio of the three plasmids was set at 1:0.4:0.01.

(3) On the next day, D-2-HG was added at concentrations of 0, 1, 2.5, 5, 10, 20, 30, 40, 50, 60, 70, and 80 μM; and a fluorescence microplate reader measured a fluorescence intensity.

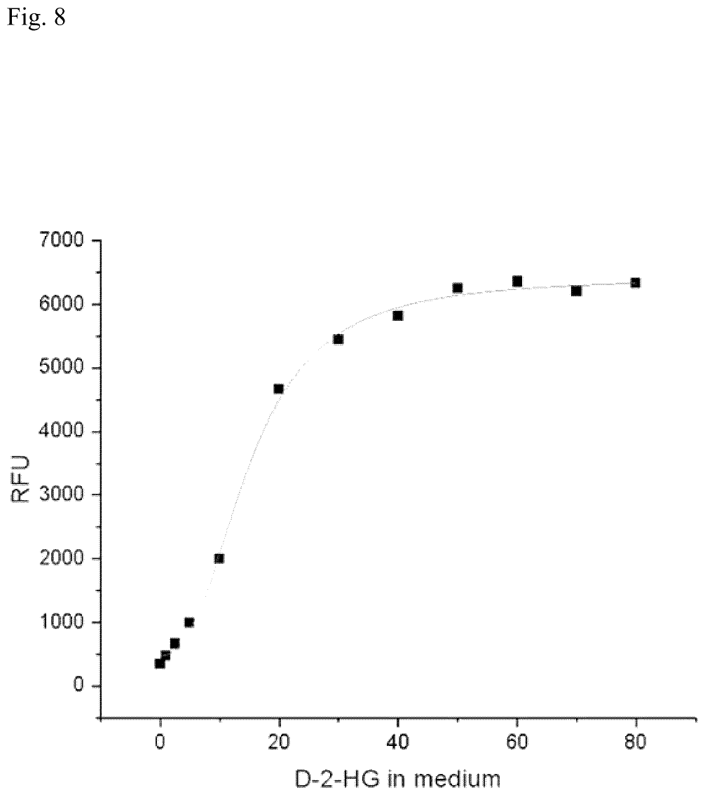

(4) As shown in FIG. 8, the control system composed of the three plasmids can sense low-concentration D-2-HG and achieve D-2-HG concentration-dependent transgene expression control.

### Example 8

### Construction of a Transposable Plasmid Carrying HGind-L

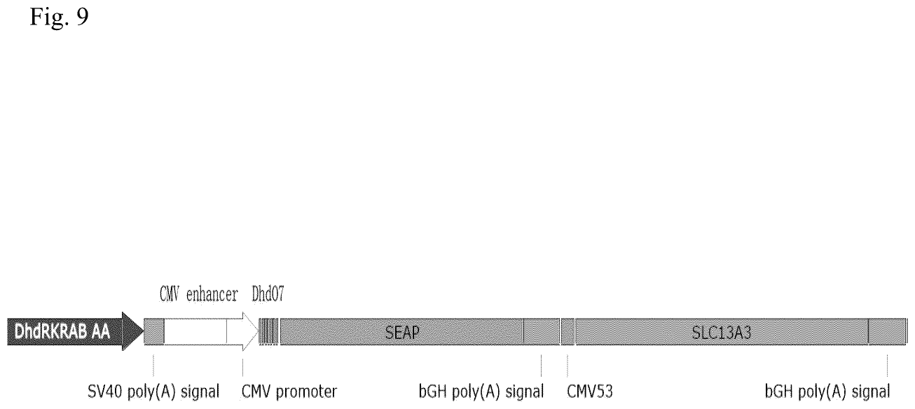

(1) Design of a long fragment A: a designed fragment was shown in FIG. 9, the sequence of which is SEQ ID NO. 17. The long fragment A includes DhdR-KRAB, SV40 poly(A) signal, CMV enhancer and promoter, Dhd07, SEAP, bGH poly(A) signal, minimal CMV promoter, SLC13A3, and bGH poly(A) signal in sequence; where a 5′ terminus includes an XbaI restriction enzyme cutting site, and a 3′ terminus includes a NotI restriction enzyme cutting site.

(2) The long fragment A was formed by whole gene synthesis.

(3) The long fragment A was linked into a PiggyBac Dual promoter plasmid through XbaI and NotI. The constructed plasmid was named PiggyBac-HGind-L, and the plasmid includes HGind-L: DhdR-KRAB itself was driven to express by a CMV promoter of the PiggyBac Dual promoter plasmid; CMV enhancer and promoter and Dhd07 constituted an inducible promoter; SEAP was a gene to be transcribed; and the D-2-HG transporter protein SLC13A3 was driven to express by a weak promoter CMV53 (sequence: SEQ ID NO.11).

### Example 9

Construction of a transposable plasmid carrying HGind-H

(1) A SLC13A3 fragment can be cut from the PiggyBac-HGind-L plasmid constructed above by KpnI single restriction digestion, and a SLC13A3-free plasmid can be obtained by self-ligation. At the same time, CMV enhancer and promoter were inserted at XbaI, and the resulting plasmid was named PiggyBac-HGind-H.

(2) The PiggyBac-HGind-H plasmid includes HGind-H: DhdR-NLS-KRAB itself was driven to express by CMV enhancer and promoter; CMV enhancer and promoter and Dhd07 constituted an inducible promoter; SEAP was a gene to be transcribed.

### Example 10

Construction of a Living Cell Sensor that Senses High-Concentration D-2-HG Based on HEK293 Cells

(1) A PiggyBac-HGind-H plasmid and a Super PiggyBac Transposase (PB200PA-1) plasmid were transfected into HEK293 cells at a ratio of 3:1 using lipofectamine 3000.

(2) After 48 hours, puromycin was added to screen for about 10 days; if necessary, monoclonal screening can be performed; and a living cell sensor 293-HGind-H was obtained.

(3) The screened cells were added to D-2-HG; and the concentrations of D-2-HG were 0 mM and 10 mM.

(4) After 72 hours, the supernatant was taken to measure SEAP.

(5) The concentration of SEAP in the supernatant without D-2-HG was about 30 U/L; and the concentration of SEAP in the supernatant with 10 mM D-2-HG was 620 U/L.

### Example 11

Construction of a Living Cell Sensor that Senses Low-Concentration D-2-HG Based on HEK293 Cells

(1) A PiggyBac-HGind-L plasmid and a Super PiggyBac Transposase (PB200PA-1) plasmid were transfected into HEK293 and HEK293FT cells at a ratio of 3:1 using lipofectamine 3000.

(2) After 48 hours, puromycin was added to screen for about 10 days; if necessary, monoclonal screening can be performed; and a living cell sensor 293-HGind-L was obtained.

(3) The screened cells were added to D-2-HG; and the concentrations of D-2-HG were 0, 20, and 40 μM.

(4) After 72 hours, the supernatant was taken to measure SEAP.

(5) The concentration of SEAP in the supernatant without D-2-HG was about 25 U/L; the concentration of SEAP in the cellular supernatant with D-2-HG (20 μM) was 750 U/L; and the concentration of SEAP in the cellular supernatant with D-2-HG (40 μM) was 1200 U/L.

### Example 12

Subcutaneous inoculation of D-2-HG living cell sensors into nude mice

(1) One group of nude mice was inoculated with HT1080 to form tumors; the other group was not inoculated with cells; HT1080 cells carried a natural IDH mutation and can produce D-2-HG.

(2) After 7 days, a living cell sensor 293-Sensor-L was subcutaneously inoculated, with 3*106 living cells per mouse.

(3) After 72 hours, blood was drawn and a SEAP assay kit measured a SEAP activity.

(4) The concentration of SEAP in the blood in a control group was about 30 mU/L, and the concentration of SEAP in the blood in the group inoculated with HT1080 was about 900 mU/L.

### Example 13

### Detection of Early-Stage Tumors in Mice by D-2-HG Living Cell Sensors

(1) SEAP in a PiggyBac-HGind-L plasmid was replaced with secretory Gaussia luciferase (Gluc) (sequence: SEQ ID NO.18) by a conventional method to obtain a plasmid PiggyBac-HGind-L-Gluc.

(2) The PiggyBac-HGind-L-Gluc and Super PiggyBac Transposase were transfected using liposomes (Lipofectamine 3000) and entered RAW264.7 macrophages, and stable transfected cells were selected with puromycin, and the resulting living cell sensor was named RAW-1.

(3) By taking a murine colon carcinoma cell CT26 as a starting cell, CT26-Fluc-IDH was constructed using a method for constructing a stable transfected cell line based on lentivirus [Nat Commun. 2021, 12 (1): 7108], the cell stably expressing an IDH1 R132H mutant protein.

(4) CT26-FLuc-IDH cells were subcutaneously inoculated into BALB/c mice aged 6-8 weeks to form tumors; the cell sensor RAW-1 was injected in the range of 0-500 mm³; according to the tumor volume (mm³), the mice were divided into 0, 0-50, 50-100, >100 groups (n=12); the cell sensor RAW-1 (1×10⁷/mouse) was injected by means of tail vein injection.

(5) 24 hours after the injection of the cell sensor, 50 μl of blood was drawn from the submandibular vein every 24 hours for 4 days, and after the blood was subjected to centrifugal treatment, a kit measured the GLuc luminescence intensity of the blood.

(6) The luminescence intensities of the groups were compared and subjected to statistical analysis, then it was concluded that the living cell sensor can effectively distinguish between tumors with a volume of 0 and 50-100 mm³.

### Example 14

### In Vivo Transfection of a Transposable Plasmid Carrying HGind-L

(1) A plasmid PiggyBac-HGind-L was mixed with a transfection reagent using an in vivo-jetPEI reagent of the Polyplus Company according to the operating instructions.

(2) The resulting mixture was incubated at room temperature for 15 minutes.

(3) Nude mice were divided into groups as in Example 12, and subjected to tail vein injection.

(4) After 72 hours, the concentration of SEAP in the blood in a control group was about 20 mU/L, and the concentration of SEAP in the blood in the group inoculated with HT1080 was about 300 mU/L.

### Example 15

### Construction and Optimization of a Lentiviral Plasmid Carrying HGind-H

(1) A lentiviral plasmid from pLenti PGK GFP Puro (w509-5) [addgene No. 19070] was used as a skeleton, and a recombinant transcriptional repressor DhdR-KRAB was placed under a mPGK promoter to replace Puro by conventional molecular cloning.

(2) A hPGK promoter was replaced with a D-2-HG inducible strong promoter by conventional molecular cloning, i.e., DhdO(n₁)-hPGK promoter-DhdO(n₂); there were combinations as follows: n₁=0 and n₂=3 (the resulting plasmid was named PGK-DHDO3); n₁=0 and n₂=7 (the resulting plasmid was named PGK-DHDO7); n₁=1 and n₂=1 (the resulting plasmid was named PGK-DHDO11); n₁=1 and n₂=2 (the resulting plasmid was named PGK-DHDO12); n₁=1 and n₂=3 (the resulting plasmid was named PGK-DHDO13); n₁=2 and n₂=1 (the resulting plasmid was named PGK-DHDO21); n₁=2 and n₂=2 (the resulting plasmid was named PGK-DHDO22); n₁=2 and n₂=3 (the resulting plasmid was named PGK-DHDO23); n₁=3 and n₂=3 (the resulting plasmid was named PGK-DHDO33). With DhdO2-hPGK promoter-DhdO2 as an example, see the sequence SEQ ID NO.19, for other sequences, DhdO sequences were added or subtracted at corresponding positions according to the number of DhdO (the n₁ and the n₂ above).

(3) The EGFP on the plasmid was replaced with a gene sequence D2eGFP to be transcribed by conventional molecular cloning.

(4) As a result, the lentiviral plasmid carried a recombination repressor DhdR-KRAB, a D-2-HG inducible strong promoter and a sequence to be transcribed, to constitute HGind-H.

(5) 293 FT cells were digested with trypsin and seeded in a 24-well plate with 1.5*105 cells per well and 500 μl complete medium (DMEM high glucose+10% FBS+1% mycillin) per well; on the next day, a corresponding plasmid was transfected at 0.75 ug per well) and a Blank group in which plasmids was not transfected was provided; 24h later, each group of cells was added to complete media with a final concentration being D-2-HG concentrations (0 and 10 mM). After 48 h, the supernatant was removed and the GFP fluorescence intensity was detected using a multifunctional microplate reader.

(6) Results showed that from the perspective of induction multiples, the induction multiples of PGK-DHDO7, PGK-DHDO13, PGK-DHDO23, and PGK-DHDO33 could reach about 8 times, which were higher than those of other combinations; and from the perspective of fluorescence intensity (fluorescent protein expression), the fluorescence intensities of PGK-DHDO13, PGK-DHDO23, and PGK-DHDO33 were relatively close, which were about 10 times those of PGK-DHDO7.

### Example 16

### Preparation of Lentivirus Carrying HGind-H and Infection of Jurkat Cells

(1) Modification of a lentiviral plasmid: based on the above PGK-DHDO33 plasmid, the recombinant transcriptional repressor DhdR-KRAB was placed under an EF1A promoter by conventional molecular cloning; and a plasmid PGK-DHDO33-D2eGFP-EF1A-DHDRKRAB was obtained.

(2) Preparation of a lentivirus
a) Preparation of a solution A: 1.2 mL of an Opti-MEM medium was added into a 1.5 mL centrifuge tube, 10 μg of the PGK-DHDO33-D2eGFP-EF1A-DHDRKRAB plasmid (with a puromycin resistance fragment), 7.5 ug of a psPAX2 plasmid, and 2.5 μg of a pMD2.G plasmid were added, and vortex oscillation was performed for 5-10 seconds to mix thoroughly;
b) Preparation of a solution B: 1.2 mL of an Opti-MEM medium was added into a 1.5 mL centrifuge tube, 100 μL of lipo3000 (50 μL of p3000+50 μL of lipo3000) was added, and vortex oscillation was performed for 5-10 seconds;
c) The solution A and the solution B were mixed at a ratio of 1:1 (v/v) and placed at room temperature for 20 minutes;
d) Preparation of 293 ft cells: 293 ft cells were cultured in a 10 cm culture dish until the cell density reached 60-70%;
e) A mixture of the solution A and the solution B at step c) was dripped evenly into the 10 cm culture dish at step d), the culture dish was stood for 3-5 minutes and then shaken gently, and cultivation was performed in a 37° C. incubator for 2 days; and
f) The resulting object was centrifuged at 300 rcf for 10 minutes to remove cell debris, the supernatant was collected and ultracentrifuged at 110,000 g for 2 hours, the supernatant was discarded, a precipitate was resuspended with sterile PBS, and stored in separate devices at −80° C.

(3) The above lentivirus was added into Jurkat cells (MOI=30).

(4) The Jurkat cells and the Jurkat cells infected with the above lentivirus were counted, and the two types of cells were respectively seeded in a 24-well plate, with 1*10⁵ cells/well, and divide into 4 groups (3 replicate wells in each group), and cultured in complete culture media with D-2-HG concentrations (0 mM, 1 mM, 5 mM, and 10 mM) for 48h.

(5) The cells were taken out for flow cytometry, and the positive rate of D2eGFP on the cells was detected using a FITC channel (Table 2).

TABLE 2
Positive rate of FITC

Positive rate of FITC per well

| | | | |
|---|---|---|---|
| Jurkat cells (lentivirus | | | |
| carrying HGind-H)) | | | |
| 0 mM | 5.78% | 5.98% | 6.47% |
| 1 mM | 8.75% | 9.02% | 9.61% |
| 5 mM | 16.42% | 16.9% | 18.91% |
| 10 mM | 22.65% | 22.04% | 21.61% |
| Jurkat cell | | | |
| 0 mM | 1.74% | 1% | 0.98% |
| 1 mM | 1.26% | 1.31% | 1.88% |
| 5 mM | 1.05% | 1.29% | 1.65% |
| 10 mM | 1.82% | 1.27% | 1.05% |

### Example 17

### Use of HGind-H in Constructing Therapeutic CAR-T Cells

(1) By conventional molecular cloning, a plasmid hPGK promoter derived from pLenti PGK GFP Puro (w509-5) [addgene No. 19070] was replaced with a D-2-HG inducible strong promoter, i.e., DhdO3-hPGK promoter-DhdO3; at the same time, a synthesized CD19-targeting BBZ-CAR fragment (sequence: SEQ ID NO.20) was placed under the control of this promoter; and the resulting plasmid was named PGK-DHDO33-BBZ-Puro.

(2) By conventional molecular cloning, a plasmid hPGK promoter derived from pLenti PGK GFP Puro (w509-5) [addgene No. 19070] was replaced with an EF1A promoter; DhdR-KRAB was placed under the EF1A promoter; a puromycin resistance gene was replaced with a hygromycin resistance gene HygR; and the resulting plasmid was named PGK-EF1A-DhdRKRAB-HygR.

(3) Similar to the above examples, PGK-DHDO33-BBZ-Puro and PGK-EF1A-DhdRKRAB-HygR were used to package lentivirus respectively.

(4) Primary human CD3+ T cells were isolated, activated and expanded using a method reported in the literature [Cytotherapy. 2021, 23 (12): 1085-1096.], at the same time, the above lentivirus was added to uniformly mix with the CD3+T cells at a ratio of MOI=30 to culture; puromycin and hygromycin were added to the culture medium starting from 72 hours until harvest; the resulting T cells are CAR-T cells carrying HGind-H and targeting CD19 antigen (HGind-H-CD19CAR-T).

(5) A HT1080 cell line has a natural IDH R132C mutation, where the IDH R132C mutation can cause the cells to secrete excessive D-2-HG. HT1080 cells that stably express CD19 antigens and firefly luciferase were constructed using the lentiviral method reported in the literature, and the resulting cells were named HT1080-CD19-Luc; HT1080 cells that stably express firefly luciferase were constructed, and the resulting cells were named HT1080-Luc.

(6) The HT1080-CD19 cells and the HT1080 cells were seeded in a 96-well plate, with 1*10⁴ cells per well. After the HT1080-CD19 cells and the HT1080 cells adhered to the wall, the supernatant was removed, and corresponding T cells were added by group, and divided into the following 4 groups: (a) HT1080-luc cells: control T cells=1:5; (b) HT1080-luc cells: HGind-H-CD19CAR-T cells=1:5; (c) HT1080-CD19-luc cells: control T cells=1:5; and (d) HT1080-CD19-luc cells: HGind-H-CD19CAR-T cells=1:5. In each group, corresponding tumor cells grown for the same time (without any T cells) were used as blank controls.

(7) After 72 hours of an action, the supernatant was removed, the cells in the well plate were digested separately, and luciferase was measured using a kit. Tumor cell survival rate (Survival rate) %=fluorescence of the experimental group-fluorescence of the corresponding blank group×100%.

(8) Results: the HT1080 cell line has an IDH R132C mutation, where the IDH R132C mutation can cause the cells to secrete excessive D-2-HG. HGind-H-CD19CAR-T cells can respond to D-2-HG in the cell supernatant to express CAR molecules targeting CD19, and can specifically kill HT1080-luc expressing CD19 antigen. After 72 hours, the killing rate of HT1080-CD19-luc by the HGind-H-CD19CAR-T cells can reach 81.3% (average value), that is, the survival rate of HT1080-CD19-luc can reach 18.7%, while the survival rate in other groups was above 60%.

### Example 18

### Use of HGind-H in Controlling Cell Expression of Cytokines and Chemokines

(1) By conventional gene synthesis and molecular cloning, the BBZ of PGK-DHDO33-BBZ-Puro in the above example was replaced with a 7-10 combination gene, i.e., IL-7 (promoting T cell proliferation) and a chemokine CXCL10 (inducing T cell infiltration [J Clin Invest. 2017, 127 (4): 1425-1437]), the two were linked by a 2A sequence (the resulting polypeptide is shown in the sequence SEQ ID NO.21), and the resulting plasmid was named PGK-DHDO33-7-10-Puro.

(2) Similar to the above example, PGK-DHDO33-7-10-Puro and PGK-EF1A-DhdRKRAB-HygR were used to package lentivirus respectively.

(3) The T cells were extracted, activated and amplified as in the above example, and the lentivirus obtained at step 2 was used to infect the T cells. Puromycin and hygromycin were added to the culture medium starting from 72 hours until harvest; the obtained T cells were T cells carrying HGind-H and controlling the expression of the cytokine IL-7 and the chemokine CXCL10 (HGind-H-7-10-T).

(4) Control T cells and HGind-H-7-10-T cells were added into 0, 5 mM D-2-HG to culture for 72h, respectively; the supernatant was taken and assayed with an ELISA kit. Compared with the control T cells, the concentrations of IL-7 and CXCL10 were upregulated by about 20 times and 14 times, respectively.

### Example 19

### Use of HGind-H in Controlling the Expression of D-2-HG Catabolic Enzymes

(1) By conventional molecular cloning, BBZ of PGK-DHDO33-BBZ-Puro in the above example was replaced with human D-2-HG dehydrogenase D2HGDH (sequence: SEQ ID NO.22), and the resulting plasmid was named PGK-DHDO33-D2HGDH-Puro;

(2) Similar to the above example, PGK-DHDO33-D2HGDH-Puro and PGK-EF1A-DhdRKRAB-HygR were used to package lentivirus respectively.

(3) T cells were extracted, activated and amplified as in the above example, and the lentivirus obtained at step (2) was used to infect the T cells. Puromycin and hygromycin were added to the culture medium starting from 72 hours until harvest; the obtained T cells were T cells carrying HGind-H and controlling the expression of D2HGDH (HGind-H-D2HGDH-T).

(4) Control T cells and HGind-H-D2HGDH-T cells were added into 0, 5 mM D-2-HG respectively to culture for 72 h; the cells were taken and then lysed, and the D2HGDH enzyme activity in the lysate was determined. Compared with the control T cells, the D2HGDH enzyme activity of the HGind-H-D2HGDH-T cells was upregulated by about 8 times.

### Example 20

### Use of HGind-H in Controlling the Expression of Suicide Genes In Vitro

(1) A lentiviral plasmid derived from pLenti PGK GFP Puro (w509-5) [addgene No. 19070] was selected as a skeleton, and a recombinant transcriptional repressor DhdR-KRAB was placed under a mPGK promoter to replace Puro by conventional molecular cloning; a hPGK promoter was replaced with a D-2-HG inducible strong promoter, i.e., DhdO(n1)-hPGK promoter-DhdO(n2); n₁=0 and n₂=14; and an EGFP gene on the plasmid was replaced with a pUΔTK gene which is a fusion gene of shortened HSV-TK and puromycin resistance genes [Nucleic Acids Res. 2004, 32 (20):e161], and the expressed fusion protein has the functions of the proteins encoded by the two genes. Thus, the lentiviral plasmid carried a recombination repressor DhdR-KRAB, a D-2-HG inducible strong promoter and a suicide gene Sui to form HGind-H-Sui; and the plasmid was named PGK-DHDO14-pUΔTK.

(2) As above conventional methods, a lentivirus carrying HGind-H-Sui was prepared using the PGK-DHDO14-pUΔTK plasmid.

(3) The human fibrosarcoma cell line HT1080 was cultured in a MEM complete medium (89% MEM+10% fetal bovine serum+1% mycillin) at 37° C. and 5% CO₂. The lentivirus prepared at step (2) was added to 5*105 HT1080 cells, where MOI=30. After the cells adhered to the wall, puromycin with a final concentration of 2 μg/ml was added to the culture medium for screening, cultivation was enlarged, and the resulting object was named HT1080-DHDO14 cell line in this patent.

(4) HT1080 cells and HT1080-DHDO14 cells were digested respectively and seeded in a 96-well plate with 3000 cells per well, a 200 μl MEM complete medium was provided. After the cells adhered to the wall, the cells were divided into six groups, and the concentrations of GCV in the culture medium were 0, 1 ng/ml, 100 ng/ml, lug/ml, 10 μg/ml, and 100 μg/ml.

(5) After 48 hours, the cytotoxicity was determined using a CCK8 kit, and viable cells (%)-(luminescence intensity of the experimental group-luminescence intensity of the control group)/luminescence intensity of the control group*100% was measured at 450 nm.

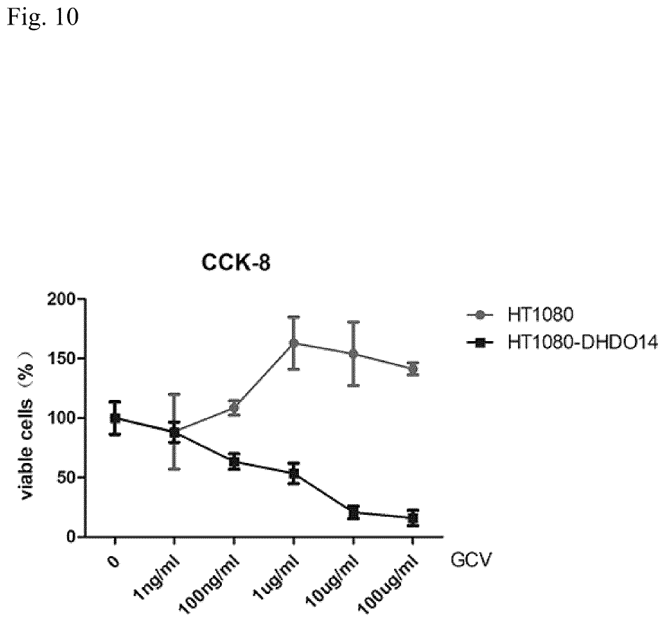

(6) Results were shown in FIG. 10. The HT1080 cell line has an IDH R132C mutation, where the IDH R132C mutation can cause the cells to secrete excessive D-2-HG, indicating that the HT1080-DHDO14 cells can respond to high-concentration D-2-HG and thus express suicide genes, and cause mutated tumor cells to die. Therefore, in vitro experiments have proven that HGind-H controls the expression of suicide genes.

### Example 21

### Use of HGind-H in Controlling the Expression of Suicide Genes in Animals

(1) 18 Balb/c nude crlj which are male and 5 weeks old were purchased. HT1080 and HT-1080-DHDO14 cells were resuspended with PBS and injected at 1.5×10⁶ cells/side*mouse (volume: 100 μl), where HT1080 was injected into the left backs of the mice and HT1080-DHDO14 was injected into the right backs of the mice.

(2) The mice were divided into two groups, the control group (n=8) was injected with normal saline, and the experimental group (n=10) was injected with ganciclovir (GCV).

(3) From the 5th to the 12th day of tumor model construction, each mouse in the control group was intraperitoneally injected with normal saline at 100 μl/day, and each mouse in the experimental group was intraperitoneally injected with GCV at 50 mg/kg per day. Starting from the 8th day, the long and short diameters of tumors of the mice were measured every day.

(4) The mice were killed on the 14th day and the tumors of the mice were weighed.

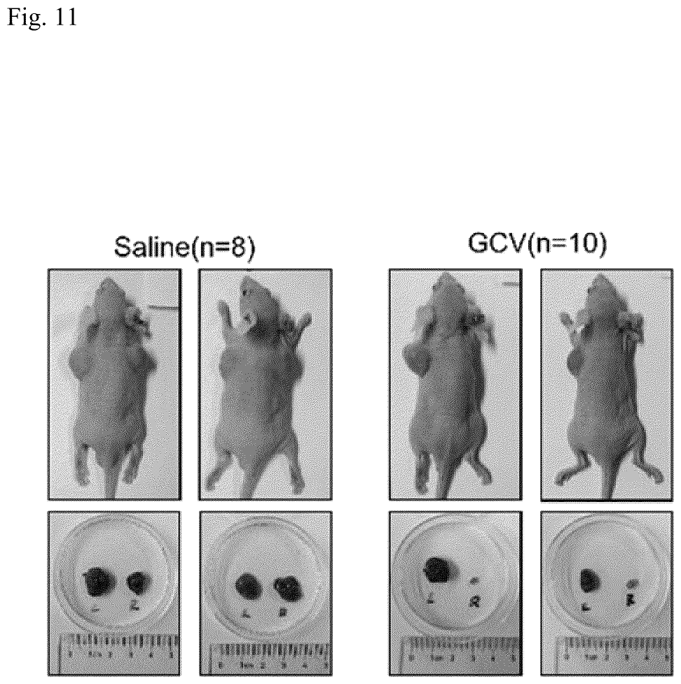

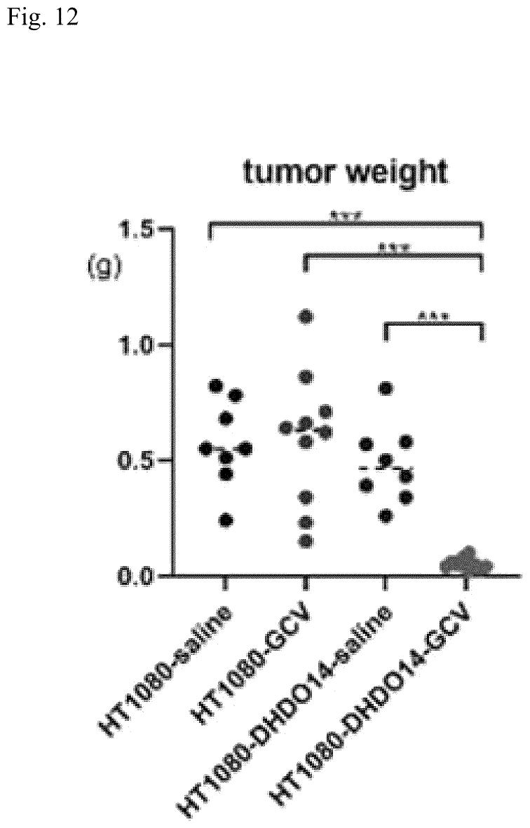

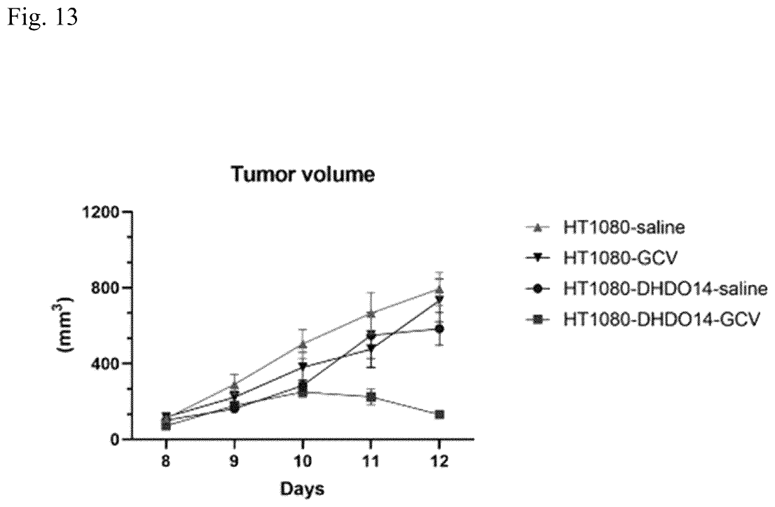

As shown in FIG. 11, the sizes of a HT1080 tumor on the left side of the mouse and a HT1080-DHDO14 tumor on the right side of the mouse in the saline group are basically the same, and the size of HT1080-DHDO14 tumor on the right side of the mouse is significantly smaller than the HT1080 tumor on the left side of the mouse in the GCV group. FIG. 12 shows that the weight of the HT1080-DHDO14 tumor in the GCV group is significantly less than that in the other 3 groups, and FIG. 13 shows that the volume of the HT1080-DHDO14 tumor in the GCV group is significantly less than that in the other three groups at the later stage of measurement. The above data shows that when the D-2-HG content in the tumor microenvironment is high, a prodrug ganciclovir can target to kill tumor cells carrying HGind and suicide genes.

### Example 22

In vivo transfection of plasmids carrying HGind-H and suicide genes

(1) A suicide gene plasmid was constructed as in Example 20, except that n1-3 and n2=3. The resulting plasmid was named PGK-DHDO33-pUΔTK;

(2) The PGK-DHDO33 and PGK-DHDO33-pUΔTK plasmids were mixed with a transfection reagent respectively using an in vivo-jetPEI reagent of Polyplus Company according to the operating instructions; and the resulting mixture was incubated at room temperature for 15 minutes;

(3) Nude mice were inoculated with HT1080 cells to form tumors and divided into 2 groups, a mixture of the plasmid at step (2) and the in vivo-jet PEI reagent was respectively injected intratumorally; the injection was performed once every 4 days for a total of 3 injections; and each mouse was intraperitoneally injected with GCV at 50 mg/kg per day.

(4) The mice were killed on the 14th day and the tumors of the mice were weighed. The tumor weight of the mice transfected with the PGK-DHDO33-pUΔTK in vivo was 30% of that of the mice transfected with PGK-DHDO33. This shows that the plasmids carrying HGind-H and suicide genes can be transfected into tumor cells by in vivo transfection reagents, and high-concentration D-2-HG is accumulated in HT1080 tumors, so that the expression of suicide genes can be induced.

1. A control system for inducing transgene expression by means of D-2-hydroxyglutarate (D-2-HG), wherein: a sensing object of the control system is D-2-HG, and the control system is classified into a control system for inducing transgene expression by means of high-concentration D-2-HG (HGind-H) and a control system for inducing transgene expression by means of low-concentration D-2-HG (HGind-L); the control system comprises a recombinant transcriptional repressor, a D-2-HG inducible promoter and a sequence to be transcribed; and
the high concentration is greater than 0.5 mmol/L, and the low concentration is less than or equal to 0.5 mmol/L.

2. The control system for inducing transgene expression by means of D-2-HG according to claim 1, wherein the recombinant transcriptional repressor is obtained by fusing a transcriptional repressor protein KRAB, a bacterial transcription repression regulatory factor DhdR that senses D-2-HG, and a nuclear localization signal NLS;
the transcriptional repressor protein KRAB is a rat zinc-finger protein Kid-1, the sequence of which is SEQ ID NO.1, or a human zinc-finger protein ZNF10, the sequence of which is SEQ ID NO.2;
the bacterial transcription repression regulatory factor DhdR that senses D-2-HG is a bacterial transcription repression regulatory protein that responds to D-2-HG, and should fulfill the following features:
DhdR has a DNA binding domain and a ligand binding domain, and in bacteria, DhdR binds to a DhdR protein-specific binding DNA sequence DhdO to prevent the transcription of a target gene, and after binding to D-2-HG, dissociates from DhdO, so that the target gene is transcribed, and DhdR and DhdO constitute a bacterial D-2-HG operon; and
the nuclear localization signal NLS is a domain that guides a protein into a cell nucleus, and the amino acid sequence of the NLS is PKKKRKV.

3. The control system for inducing transgene expression by means of D-2-HG according to claim 1, wherein the D-2-HG inducible promoter is formed by tandem connection of a constitutive promoter Pc and a DhdR protein-specific binding DNA sequence DhdO, DhdO being located upstream or/and downstream of Pc; and the D-2-HG inducible promoter is abbreviated as DhdO(n₁)-Pc-DhdO(n₂), wherein n₁ and n₂ are the numbers of tandem repeats of DhdO, 0≤n₁≤14, 0≤n₂≤14, and n₁ and n₂ are not 0 simultaneously; and
Pc is a constitutive promoter in a eukaryotic cell, and comprises, but is not limited to, CMV, hPGK, mPGK or EF1α.

4. The control system for inducing transgene expression by means of D-2-HG according to claim 1, wherein the sequence to be transcribed comprises, but is not limited to, secretory alkaline phosphatase, Gaussia luciferase, firefly luciferase, an enhanced fluorescent protein, a chimeric antigen receptor, a cytokine, a chemokine, a suicide gene or a D-2-HG catabolic enzyme.

5. The control system for inducing transgene expression by means of D-2-HG according to claim 1, wherein when the control system is a control system for inducing transgene expression by means of low-concentration D-2-HG (HGind-L), the control system further comprises a D-2-HG transporter protein;
the D-2-HG transporter protein is a SLC13A3 protein, and the SLC13A3 protein is driven to express by a weak promoter or minimal promoter;
the weak promoter or minimal promoter comprises, but is not limited to, a minimal CMV promoter, a mini-TK promoter or CMV53.

6. A construction method for the control system for inducing transgene expression by means of D-2-HG according to claim 1, characterized by comprising the following steps:
(1) a control system for inducing transgene expression by means of D-2-HG was designed and synthesized, wherein the system comprises a recombinant transcriptional repressor, a D-2-HG inducible promoter, and a sequence to be transcribed;
(2) a vector carrying the control system for inducing transgene expression by means of D-2-HG was prepared, wherein the vector is a eukaryotic plasmid expression vector, a viral particle or a transfection reagent;
(3) the vector carried the control system for inducing transgene expression by means of D-2-HG to enter a target cell, wherein the target cell is a cell line or a primary cell, and the primary cell comprises, but is not limited to, a tumor cell or an immune cell; and
(4) the target cell sensed the concentration of D-2-HG and then induced transgene expression.

7. The construction method for the control system for inducing transgene expression by means of D-2-HG according to claim 6, wherein when the control system is a control system for inducing transgene expression by means of low-concentration D-2-HG (HGind-L), when a control system for inducing transgene expression by means of D-2-HG was designed and synthesized at step 1, a D-2-HG transporter protein needs to be added.

8. Application of the control system for inducing transgene expression by means of D-2-HG described in claim 1 in constructing a D-2-HG living cell sensor, and application of the living cell sensor in vitro and in vivo.

9. Application of the control system for inducing transgene expression by means of D-2-HG described in claim 1 in constructing a suicide gene therapy vector and application of the suicide gene therapy vector in a tumor suicide gene therapy.

10. Application of the control system for inducing transgene expression by means of D-2-HG described in claim 1 in constructing a therapeutic cell and application of the therapeutic cell in tumor treatment.

11. A construction method for the control system for inducing transgene expression by means of D-2-HG according to claim 2, characterized by comprising the following steps:
(1) a control system for inducing transgene expression by means of D-2-HG was designed and synthesized, wherein the system comprises a recombinant transcriptional repressor, a D-2-HG inducible promoter, and a sequence to be transcribed;
(2) a vector carrying the control system for inducing transgene expression by means of D-2-HG was prepared, wherein the vector is a eukaryotic plasmid expression vector, a viral particle or a transfection reagent;
(3) the vector carried the control system for inducing transgene expression by means of D-2-HG to enter a target cell, wherein the target cell is a cell line or a primary cell, and the primary cell comprises, but is not limited to, a tumor cell or an immune cell; and
(4) the target cell sensed the concentration of D-2-HG and then induced transgene expression.

12. A construction method for the control system for inducing transgene expression by means of D-2-HG according to claim 3, characterized by comprising the following steps:
(1) a control system for inducing transgene expression by means of D-2-HG was designed and synthesized, wherein the system comprises a recombinant transcriptional repressor, a D-2-HG inducible promoter, and a sequence to be transcribed;
(2) a vector carrying the control system for inducing transgene expression by means of D-2-HG was prepared, wherein the vector is a eukaryotic plasmid expression vector, a viral particle or a transfection reagent;
(3) the vector carried the control system for inducing transgene expression by means of D-2-HG to enter a target cell, wherein the target cell is a cell line or a primary cell, and the primary cell comprises, but is not limited to, a tumor cell or an immune cell; and
(4) the target cell sensed the concentration of D-2-HG and then induced transgene expression.

13. A construction method for the control system for inducing transgene expression by means of D-2-HG according to claim 4, characterized by comprising the following steps:
(1) a control system for inducing transgene expression by means of D-2-HG was designed and synthesized, wherein the system comprises a recombinant transcriptional repressor, a D-2-HG inducible promoter, and a sequence to be transcribed;
(2) a vector carrying the control system for inducing transgene expression by means of D-2-HG was prepared, wherein the vector is a eukaryotic plasmid expression vector, a viral particle or a transfection reagent;
(3) the vector carried the control system for inducing transgene expression by means of D-2-HG to enter a target cell, wherein the target cell is a cell line or a primary cell, and the primary cell comprises, but is not limited to, a tumor cell or an immune cell; and
(4) the target cell sensed the concentration of D-2-HG and then induced transgene expression.


SEQUENCE LISTING (22 sequences)

SEQ ID NO: 1 - protein, 42 aa, Rattus norvegicus
VSVTFEDVAVLFTRDEWKKLDLSQRSLYREVMLENYSNLASM

SEQ ID NO: 2 - protein, 65 aa, Homo sapiens
RTLVTFKDVFVDFTREEWKLLDTAQQIVYRNVMLENYKNLVSLGYQLTKPDVILRLEKGEEPWLV

SEQ ID NO: 3 - protein, 238 aa, Achromobacter denitrificans
  source 1..238: note = NBRC 15125
MLSKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARQTS

SEQ ID NO: 4 - protein, 239 aa, Achromobacter xylosoxidans
  source 1..239: note = ATCC 27061
MLTKSLTLTEQVARQIADDIAHGVYPVGAKLPSGRILAEQYGVSAAVIREVTERLRAQGLIQSRQGSGCVVLARTGAQGFQVPVDIAVSREQLASVYELRMELEGGAAALAAERRNAADLDALAGALADLERHQHHPEHGVEHDIAFHLAIAAATHNPFYQDLLQYLNLQLRLAVSTARANSRQLAGMTDAVHHEHVAVFEAIRDRDAARARLTAERHLRQAASRLRLDSLFPASRQTS

SEQ ID NO: 5 - protein, 310 aa, synthetic construct
MLSKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARQTSPKKKRKLVAVSVTFEDVAVLFTRDEWKKLDLSQRSLYREVMLENYSNLASMAGFLFTKPKVISLLQQGEDPW

SEQ ID NO: 6 - protein, 320 aa, synthetic construct
MHATPKKKRKLVAVSVTFEDVAVLFTRDEWKKLDLSQRSLYREVMLENYSNLASMAGFLFTKPKVISLLQQGEDPWGSGSGSMLSKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARQTS

SEQ ID NO: 7 - protein, 389 aa, synthetic construct
MLTKSLTLTEQVARQIADDIAHGVYPVGAKLPSGRILAEQYGVSAAVIREVTERLRAQGLIQSRQGSGCVVLARTGAQGFQVPVDIAVSREQLASVYELRMELEGGAAALAAERRNAADLDALAGALADLERHQHHPEHGVEHDIAFHLAIAAATHNPFYQDLLQYLNLQLRLAVSTARANSRQLAGMTDAVHHEHVAVFEAIRDRDAARARLTAERHLRQAASRLRLDSLFPASRQTSVDAPDPKKKRKVDPKKKRKVDPKKKRKVMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLW

SEQ ID NO: 8 - protein, 602 aa, synthetic construct
MAALAAAAKKVWSARRLLVLLFTPLALLPVVFALPPKEGRCLFVILLMAVYWCTEALPLSVTALLPIVLFPFMGILPSNKVCPQYFLDTNFLFLSGLIMASAIEEWNLHRRIALKILMLVGVQPARLILGMMVTTSFLSMWLSNTASTAMMLPIANAILKSLFGQKEVRKDPSQESEENTAAVRRNGLHTVPTEMQFLASTEAKDHPGETEVPLDLPADSRKEDEYRRNIWKGFLISIPYSASIGGTATLTGTAPNLILLGQLKSFFPQCDVVNFGSWFIFAFPLMLLFLLAGWLWISFLYGGLSFRGWRKNKSEIRTNAEDRARAVIREEYQNLGPIKFAEQAVFILFCMFAILLFTRDPKFIPGWASLFNPGFLSDAVTGVAIVTILFFFPSQRPSLKWWFDFKAPNTETEPLLTWKKAQETVPWNIILLLGGGFAMAKGCEESGLSVWIGGQLHPLENVPPALAVLLITVVIAFFTEFASNTATIIIFLPVLAELAIRLRVHPLYLMIPGTVGCSFAFMLPVSTPPNSIAFASGHLLVKDMVRTGLLMNLMGVLLLSLAMNTWAQTIFQLGTFPDWADMYSVNVTALPPTLANDTFRTL

SEQ ID NO: 9 - DNA, 39 bp, Cytomegalovirus
  source 1..39: mol_type = other DNA
ggtaggcgtgtacggtgggaggtctatataagcagagct

SEQ ID NO: 10 - DNA, 63 bp, Herpes simplex virus
  source 1..63: mol_type = other DNA
ttcgcatattaaggtgacgcgtgtggcctcgaacaccgagcgaccctgcagcgacccgcttaa

SEQ ID NO: 11 - DNA, 76 bp, Cytomegalovirus
  source 1..76: mol_type = other DNA
caacaaaatgtcgtaacaagggcggtaggcgtgtacggtgggaggtctatataagcagagctcgtttagtgaaccg

SEQ ID NO: 12 - DNA, 1078 bp, synthetic construct
  source 1..1078: mol_type = other DNA
ggatccaagttatcagataacctaagttatcagataacctaagttatcagataacgaattcctaagttatcagataacctaagcggccgcgttatcagataacctaagatatcctaagttatcagataacctaagttatcagataacgcggccgcctaagttatcagataacctaagttatcagataacctaagttatcagataacgaattcctctcgagcatatgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtagtctaga

SEQ ID NO: 13 - DNA, 927 bp, synthetic construct
  source 1..927: mol_type = other DNA
ggatccaagttatcagataacctaagttatcagataacctaagttatcagataacgaattcctctcgagcatatgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtagtctaga

SEQ ID NO: 14 - DNA, 1013 bp, synthetic construct
  source 1..1013: mol_type = other DNA
ggatccaagttatcagataacctaagttatcagataacctaagttatcagataacgaattcctaagttatcagataacctaagcggccgcctaagttatcagataacctaagttatcagataacctaagttatcagataacgaattcctctcgagcatatgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtagtctaga

SEQ ID NO: 15 - DNA, 1148 bp, synthetic construct
  source 1..1148: mol_type = other DNA
ggatccaagttatcagataacctaagttatcagataacctaagttatcagataacgaattcctaagttatcagataacctaagcggccgcgttatcagataacctaagatatcgttatcagataacctaagttatcagataacctaagttatcagataacctaagttatcagataacgatatcctaagttatcagataacctaagttatcagataacgcggccgcctaagttatcagataacctaagttatcagataacctaagttatcagataacgaattcctctcgagcatatgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtagtctaga

SEQ ID NO: 16 - protein, 501 aa, Homo sapiens
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTD

SEQ ID NO: 17 - DNA, 5670 bp, synthetic construct
  source 1..5670: mol_type = other DNA
tctagagccaccatgctgtctaaaagcctgactctgactgaacaggtggctagacagattgctggagatattgctgaaggagttcatagtgtgggagccaaacttcctcctggaagagtgctggctgaacagtacggagtgagcgctgccgtgattagagaggccacagaaagactgagagcccagggactgattcagagtagacagggaagcggtagcgtggtggtgagtagaactggagcacagggatttcaggtgagcgccggacttgatgatagagagcagttagcttctgtgtatgaactgagaatggaacttgaaggtggagcagccgcactggcagctagaaggagaaatgctactgatctggccgctatggccgaagctctggccgccttagaagctaatctggatcatcctgaacagggagttgaacatgacattgcttttcatgtggccattgccgctgccacacataatagatattatcaggatcttctgcagtatctgaatctgcagctgagattggccgtgagcacagctagaacaaatagcagaagacaggaaggactgacagccgtggtgcatcaggagcatgtggcagtctatgatgccattctggctggagatcctgatagagctagactggctgccacaagacatctgcagcaggccgcctccaggctgcggctggacctgctgagcccagccgccaggcagaccagccccaagaagaagagaaagctggccgtgagcgtgacctttgaggacgtggccgtgctgttcacaagggacgagtggaagaagctggacctgagccagagaagcctgtacagagaagtgatgctggagaactacagcaacctggccagcatggccggcttcctgtttaccaagcccaaagtgattagcctgctgcagcagggcgaggacccctggtgaaacttgtttattgcagcttataatggttacaaataaagcaatagcatcacaaatttcacaaataaagcatttttttcactgcattctagttgtggtttgtccaaactcatcaatgtatcttagaattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctaagttatcagataacctaagttatcagataacctaagttatcagataacctaagttatcagataacctaagttatcagataacctaagttatcagataacctaagttatcagataacctgctagcgccaccatgctgctgctgctgctgctgctgggactgcggcttcaactgagcctgggcatcatccccgtggaagaagagaaccctgacttctggaaccgggaagccgccgaggccctgggcgccgctaagaagctgcaacctgcccagaccgccgccaagaacctgatcatcttcctgggagatggaatgggcgtgtctactgtgaccgctgcgagaatcctgaaaggccagaagaaggacaaactgggcccagaaatccctctcgccatggatcgctttccatacgtggccctgtctaagacctacaacgtggacaagcacgtgccagacagcggcgctacagccacagcctacctgtgcggcgtcaagggcaacttccagaccatcggcctgagcgccgctgccagattcaaccagtgcaacaccaccagaggaaacgaggtgatctccgttatgaaccgggccaagaaagccggcaagagcgtgggcgtggtgacaaccacaagagtgcaacacgccagccctgccggcacctacgcccacaccgtgaacagaaattggtacagcgacgcagatgtgcccgcctctgcccggcaggagggatgtcaggacattgccacacagctgatcagcaacatggacattgacgtgatcctgggcggaggcagaaagtatatgttcagaatgggaacccctgatcctgagtaccctgacgactattctcagggcggcacaagactggacggcaagaatctggtccaggagtggctggccaaaagacaaggagctcggtacgtgtggaatcgcaccgagctgatgcaggccagcctggaccctagcgtgacacacctgatgggactgttcgagcctggcgacatgaagtacgagatccacagggatagcaccctcgacccctccctgatggaaatgaccgaggccgccctgagactgctgagcagaaaccccagaggttttttcctgtttgtggaaggcggcagaatcgaccacggccaccacgagagcagagcctaccgggccctgaccgagacaatcatgttcgacgacgccatcgaacgggctggccagctgacaagcgaggaagataccctgagcctggtgaccgccgatcacagccatgtgttcagcttcggaggctaccccctgagaggcagcagcatctttggcctggctcctggcaaagcccgggacagaaaggcctataccgtgctgctctacggcaacggccctgggtacgtgctgaaggacggcgccagacccgacgtgaccgagtctgaatctggctcccccgagtacagacagcagtccgctgttcctctggatgaggaaacccacgccggcgaggatgtcgccgtgttcgcccggggcccccaggctcatctggtgcacggtgtgcaggagcagacattcatcgcccacgtgatggccttcgccgcatgcctggaaccttacaccgcatgcgacctggcccctcctgctggcaccacagattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggtaccgtcgaccaacaaaatgtcgtaacaagggcggtaggcgtgtacggtgggaggtctatataagcagagctcgtttagtgaaccgggatccgccaccatggccgccctggccgccgccgccaagaaggtctggtccgccagaaggctgctggtcctcctgttcacacccctggccctgctccccgtggtgttcgccctgccccccaaggagggccggtgcctgttcgtgatcctcctgatggccgtgtactggtgcacagaggccctgccactgagcgtgacagccctgctgcccatcgtgctgttccccttcatgggcattctgcccagcaacaaggtctgcccccagtacttcctggacacaaactttctgtttctgtccggcctgatcatggcctccgccatcgaggagtggaacctgcaccggaggattgccctgaagatcctgatgctggtcggggtgcagcccgccagactgattctggggatgatggtgacaacctccttcctgagcatgtggctgagcaacaccgccagcactgccatgatgctgcctattgctaacgccattctcaagtccctcttcggccagaaggaggtgaggaaggaccccagccaggagtccgaggagaacaccgccgccgtcaggcggaacgggctgcacaccgtgcccaccgagatgcagttcctcgccagcacagaggccaaggatcaccctggcgagaccgaggtgcccctggatctccccgccgatagccggaaggaggatgagtaccggcggaacatctggaagggcttcctgattagcatcccctactccgccagcattggggggaccgccacactgaccgggaccgcccccaacctcatcctcctgggccagctgaagagcttcttcccccagtgcgacgtggtgaactttgggagctggttcatcttcgccttccctctgatgctcctgttcctgctggccggctggctgtggattagcttcctgtacggggggttgagctttagggggtggcggaagaacaagagcgagatccggacaaacgccgaggatagggccagggccgtgatccgggaggagtaccagaacctgggacctattaagttcgccgagcaggccgtgttcattctgttctgcatgtttgccatcctcctcttcacacgggaccctaagttcatccccggctgggcctccctcttcaaccccggcttcctcagcgacgccgtgaccggggtcgccattgtgaccattctgttcttctttcccagccagcggccctccctcaagtggtggttcgattttaaggcccctaacacagagactgagcctctgctgacatggaagaaggcccaggagacagtgccttggaacatcatcctcctcctggggggggggttcgccatggccaagggctgcgaggagtccggcctgagcgtgtggatcggggggcagctgcatcccctggagaacgtgccccccgccctggccgtgctcctgattacagtcgtgatcgccttcttcactgagttcgccagcaacacagccactattatcatctttctgcccgtgctggccgagctggccattagactgagggtgcaccccctgtacctgatgatccccggaaccgtggggtgctccttcgccttcatgctgcccgtgagcacaccccccaactccatcgccttcgcctccggccacctgctggtgaaggatatggtgaggaccgggctgctgatgaacctgatgggcgtgctgctgctgagcctcgccatgaacacctgggcccagaccatcttccagctggggaccttccccgactgggccgacatgtacagcgtgaacgtgaccgccctgcccccaaccttggccaacgacacattccggacactgtaactcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggtaccgcggccgc

SEQ ID NO: 18 - protein, 185 aa, Gaussia princeps
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 19 - DNA, 591 bp, synthetic construct
  source 1..591: mol_type = other DNA
aagttatcagataacctaagttatcagataacctgtcgacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggatccaagttatcagataacctaagttatcagataacct

SEQ ID NO: 20 - DNA, 1479 bp, synthetic construct
  source 1..1479: mol_type = other DNA
atgctcctgctggtgacatccttgctgctgtgcgagctgcctcatcctgcgttcctgctcatcccggacatccagatgacccagacgacctcttcgctgagcgcaagcctgggagacagggtcaccatttcatgccgagcttctcaagacatctctaagtacctcaattggtatcagcagaagcccgacggcaccgtgaagctactcatttaccacacttcgcgcctgcactcgggggtgccctccaggttttcaggctctggcagtggcaccgactactccctgaccattagtaacctggaacaggaggacatcgcgacatatttctgtcaacagggcaacacgctgccgtacaccttcggcggtggcaccaagctggagatcaccggctccacgtccggctccggcaagcctggttccggggagggctccactaaaggagaggtgaagctccaggagagcgggcccggcctggtggctccctcccagagcttgtccgtcacctgtactgtgagtggcgtctcgcttcccgactacggtgttagttggattcgccagcccccccgcaaagggctggagtggctgggcgtgatttggggcagcgagaccacctattacaacagcgcgctgaagtctcggctgaccatcatcaaggacaactccaagtcccaggtcttcctgaaaatgaactctctccagaccgatgacacggccatctattactgtgccaagcactactactacggcggcagctacgccatggattattggggccaaggaaccagcgtgaccgtgtcctctgcttctacgaccaccccggcgccccgccctccaactccggctcctaccatcgcgtctcagcctttatccctgagacccgaagcatgccgtccagctgccggcggtgccgtgcacactcgcggcttggattttgcctgcgacatctacatctgggcccccctggctggcacctgcggggtactgctactcagccttgttatcactttgtactgtaagcgcggccgtaagaagctgctttacatcttcaagcagccgttcatgcgtccagtgcagacaacccaggaggaagacggctgctcttgtcgcttccccgaggaggaggagggcgggtgcgagctgcgcgtgaaatttagccgctccgcggacgctcctgcctatcagcagggacagaaccagctgtacaacgaactcaacctgggccgtcgcgaggagtacgatgtgctggacaaacgtcgcgggcgcgacccggagatgggcggaaagccgcgccgcaaaaatcctcaggagggcttgtacaacgagctacagaaggacaagatggccgaggcctactcggagatcggtatgaagggcgagcgcagacggggaaaggggcatgacgggctttatcagggtctttcgaccgctactaaggacacctacgacgcactccacatgcaggccctgcccccacggtaa

SEQ ID NO: 21 - protein, 294 aa, synthetic construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHATNFSLLKQAGDVEENPGPMNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 22 - protein, 521 aa, Homo sapiens
MLPRRPLAWPAWLLRGAPGAAGSWGRPVGPLARRGCCSAPGTPEVPLTRERYPVRRLPFSTVSKQDLAAFERIVPGGVVTDPEALQAPNVDWLRTLRGCSKVLLRPRTSEEVSHILRHCHERNLAVNPQGGNTGMVGGSVPVFDEIILSTARMNRVLSFHSVSGILVCQAGCVLEELSRYVEERDFIMPLDLGAKGSCHIGGNVATNAGGLRFLRYGSLHGTVLGLEVVLADGTVLDCLTSLRKDNTGYDLKQLFIGSEGTLGIITTVSILCPPKPRAVNVAFLGCPGFAEVLQTFSTCKGMLGEILSAFEFMDAVCMQLVGRHLHLASPVQESPFYVLIETSGSNAGHDAEKLGHFLEHALGSGLVTDGTMATDQRKVKMLWALRERITEALSRDGYVYKYDLSLPVERLYDIVTDLRARLGPHAKHVVGYGHLGDGNLHLNVTAEAFSPSLLAALEPHVYEWTAGQQGSVSAEHGVGFRKRDVLGYSKPPGALQLMQQLKALLDPKGILNPYKTLPSQA